(19) United States
(12) Patent Application Publication
(10) Pub. No.: US 2009/0005791 A1
(43) Pub. Date: Jan. 1, 2009

(54) HAIR REMOVING DEVICE

(75) Inventors: Takeshi MATSUSAKA, Hikone (JP); Hidekazu SUEYOSHI, Higashiomi (JP)

Correspondence Address: GREENBLUM & BERNSTEIN, P.L.C., 1950 ROLAND CLARKE PLACE, RESTON, VA, 20191, US

(73) Assignee: MATSUSHITA ELECTRIC WORKS, LTD., Osaka (JP)

(21) Appl. No.: 12/144,038

(22) Filed: Jun. 23, 2008

(30) Foreign Application Priority Data
Jun. 27, 2007 (JP) 2007-169277

(51) Int. Cl. A61B 17/50

(52) U.S. Cl. 606/133

(57) ABSTRACT

A notch is provided between a cylinder supporting unit and a body of a pedestal, thereby applying elasticity to the cylinder supporting unit. With this structure, a leaf spring and the cylinder supporting unit corresponding to the leaf spring can always be in contact with or approach each other, and a noise generated when the cylinder supporting unit and the body of the pedestal collides against each other can be suppressed.

### BACKGROUND OF THE INVENTION

1. Field of the Invention

The present invention relates to a hair removing device for removing hair.

2. Description of the Related Art

There have been conventionally known hair removing devices that remove hair with openable and closable hair-removing pawls sandwiching hair and pulling out the hair. Japanese Patent Application Laid-open No. 2000-201725 discloses one example of such hair removing devices. In the hair removing device disclosed in this publication, an open/close lever is rocked by a cam, and the hair-removing pawl which moves in tandem with the open/close lever is opened and closed, thereby switching the sandwiching and the release of hair. The cam presses the open/close lever by an elastic force of a leaf spring.

According to the device disclosed in Japanese Patent Application Laid-open No. 2000-201725, however, the leaf spring collides against a stopper that holds the cam, and causes a noise in some cases. Specifically, in a state where a cam 29 presses an open/close lever 39 as shown in FIG. 1A, a leaf spring 120 is pushed by a rotation shaft 29j of the cam 29 and separated from a stopper 125′, while in a state where the cam 29 does not press the open/close lever 39 as shown in FIG. 1B, the leaf spring 120 approaches or comes into contact with the stopper 125′ due to its elasticity. At that time, when the leaf spring 120 collides against the stopper 125′, a noise is generated. To reduce such a noise, it is effective that a distance between the leaf spring and the stopper is always maintained small. However, if the machining precision of parts is enhanced for that purpose, the manufacturing cost is increased.

Therefore, it is an object of the present invention to provide a hair removing device capable of reducing a noise without largely increasing its cost.

### SUMMARY OF THE INVENTION

The present invention provides a hair removing device comprising a substantially cylindrical rotation cylinder that rotates around a rotation axis, a fixing pawl that is fixed to the rotation cylinder and that has a tip end exposed from an outer periphery of the rotation cylinder, a movable pawl that can rock in an axial direction of the rotation axis, that has a tip end exposed from the outer periphery of the rotation cylinder, and that is configured such that a state where the tip end of the movable pawl abuts against the tip end of the fixing pawl and a state where these tip ends are separated are switched by a rocking motion of the movable pawl, an open/close lever that is inserted into the rotation cylinder such that the open/close lever can reciprocate in an axial direction of the rotation cylinder, and that rocks the movable pawl, a cam that reciprocates the open/close lever in the axial direction while rotating around the rotation axis, a pedestal that rotatably supports the rotation cylinder and the cam, and a spring that biases the cam toward the open/close lever to press the cam against the open/close lever, wherein a cylinder supporting unit as a portion of the pedestal that supports the rotation cylinder and the cam is can rock in association with the spring with respect to a body of the pedestal.

According to the present invention, the cylinder supporting unit can have elasticity.

According to the present invention, a notch can be formed between the cylinder supporting unit and the body.

According to the present invention, the cam can have a roller-like shape.

According to the present invention, the cylinder supporting unit can be formed with a through hole in which a rotation shaft of the cam is rotatably accommodated and the rotation shaft is biased by the spring from a side opposite from the open/close lever.

According to the present invention, a hair removing device that is improved to reduce noises resulting from collision of a leaf spring against a stopper (cylinder supporting unit) can be obtained.

### BRIEF DESCRIPTION OF THE DRAWINGS

FIG. 1A is a schematic diagram showing a motion of a stopper and a leaf spring of a conventional hair removing device, and shows a state where a cam presses an open/close lever;

FIG. 1B is another schematic diagram showing a motion of the stopper and the leaf spring of the conventional hair removing device, and shows a state where the cam releases the open/close lever;

FIG. 2A is a side view of a hair removing head of a hair removing device according to an embodiment of the present invention;

FIG. 2B is a front view of the hair removing head of the hair removing device according to the embodiment;

FIG. 3 is a sectional view taken along the line III-III in FIG. 2B;

FIG. 4 is a sectional view of the hair removing head of the hair removing device according to the embodiment taken along a plane which is parallel to a longitudinal direction of the head;

FIG. 5A is a perspective view showing a structure of the hair removing head of the hair removing device according to the embodiment;

FIG. 5B is another perspective view showing the structure of the hair removing head of the hair removing device according to the embodiment, in a state where a blade cover is removed;

FIG. 5C is another perspective view showing the structure of the hair removing head of the hair removing device according to the embodiment, and shows a hair removing block and a drive unit;

FIG. 6 is a sectional view taken along a plane including a rotation axis of a rotation cylinder;

FIG. 7 is a partial exploded perspective view of the rotation cylinder;

FIG. 8 is another partial exploded perspective view of the rotation cylinder;

FIG. 9A is a partial exploded perspective view of the hair removing head, and shows the hair removing block;

FIG. 9B is another partial exploded perspective view of the hair removing head, and shows a pedestal portion;

FIG. 10 is an exploded perspective view of the hair removing head of the hair removing device according to the embodiment;

FIG. 11A is a schematic diagram showing a motion of a cylinder supporting unit and a leaf spring of the hair removing device according to the embodiment, and shows a state where a cam presses an open/close lever; and

FIG. 11B is another schematic diagram showing a motion of the cylinder supporting unit and the leaf spring of the hair removing device according to the embodiment, and shows a state where the cam releases the open/close lever.

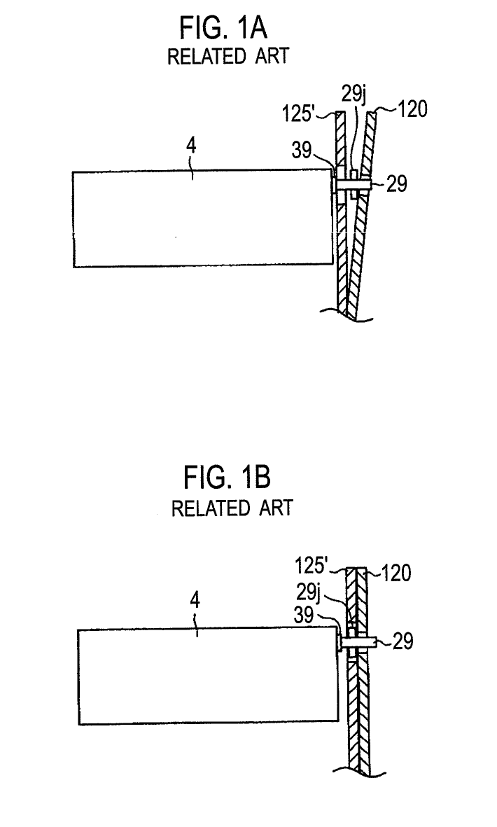

### DETAILED DESCRIPTION OF THE PREFERRED EMBODIMENTS

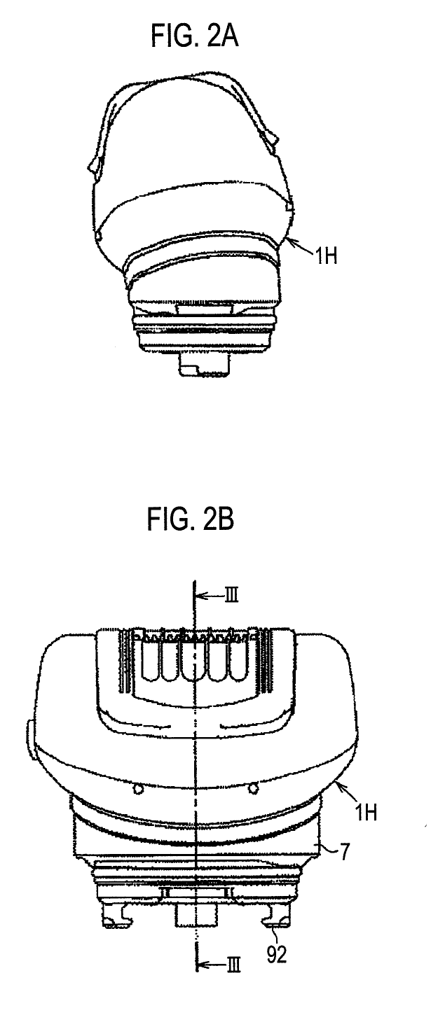

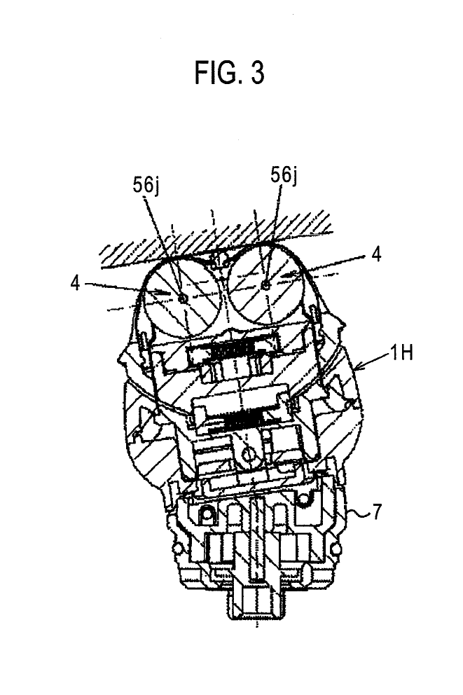

A hair removing device according to an embodiment of the present invention is explained below in detail with reference to FIGS. 2A to 11B.

The hair removing device is formed into a size such that it can be grasped by a hand, and has a body casing (not shown) in which a motor as a drive source is incorporated. As shown in FIGS. 2A to 5C, above the body casing, there is provided a hair removing head 1H in which a hair removing block 2 including two rotation cylinders 4 and a drive unit that rotates the rotation cylinders 4 are installed.

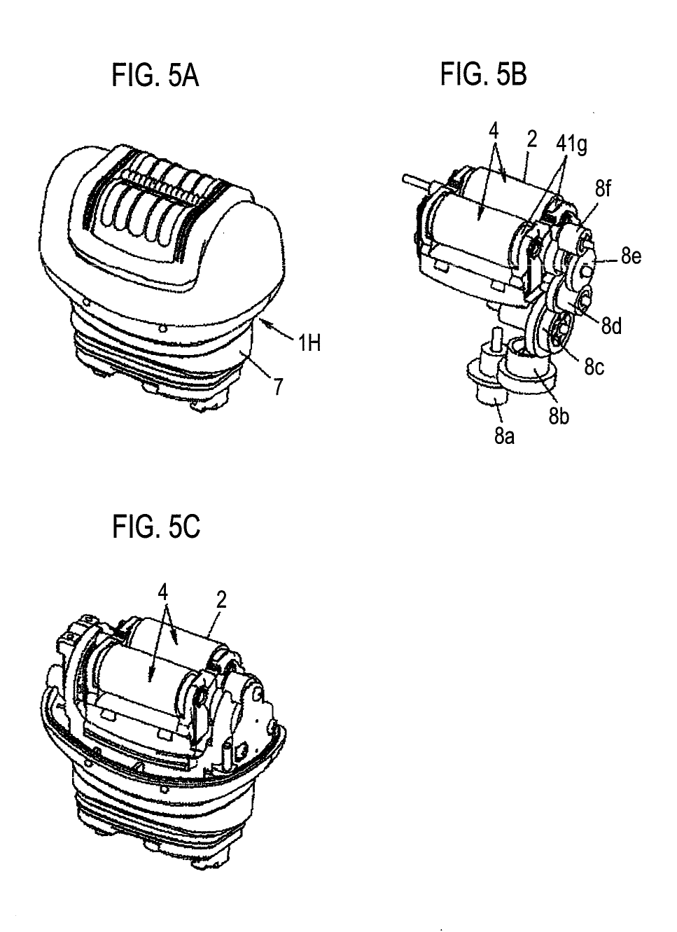

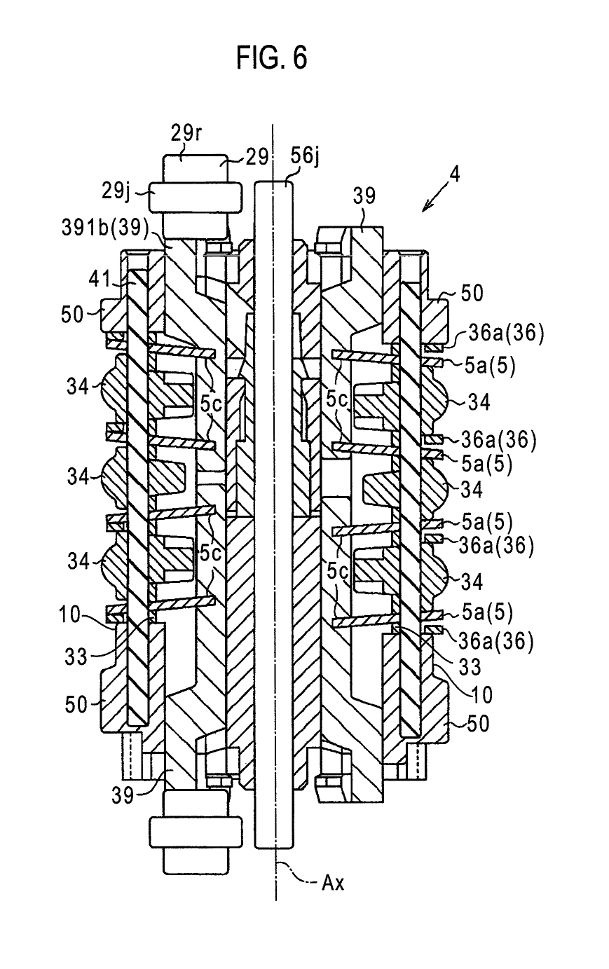

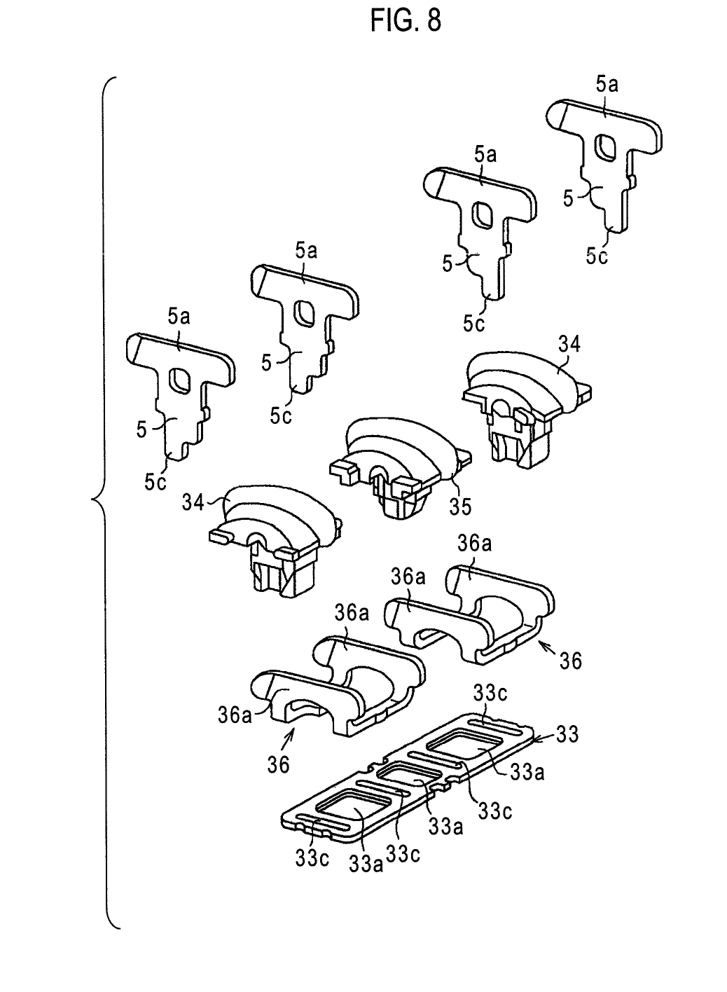

As shown in FIGS. 5A to 7, each of the rotation cylinders 4 included in the hair removing head 1H is substantially cylindrical in shape. Both ends of each rotation cylinder 4 in its axial direction are formed with end-face forming members 50 substantially of disk shapes. The end-face forming member 50 is formed at its central portion with a through hole 4b, and a shaft 56j is fitted into the through hole 4b. Eight through holes 4c are radially formed outside the through hole 4b in its circumferential direction, and an open/close lever 39b is slidably inserted into each of the through holes 4c. Eight through holes 4d are provided outside the eight through holes 4c in the circumferential direction, and a substantially columnar fixing shaft 41 is fitted into each of the through holes 4d. A projection having axial grooves 4a is formed on an inner surface of the end-face forming member 50 of the rotation cylinder at locations corresponding to the eight through holes 4d. A substantially rectangular frame-like fulcrum plate 33 as shown in FIGS. 6 and 8 is fitted into each groove 4a.

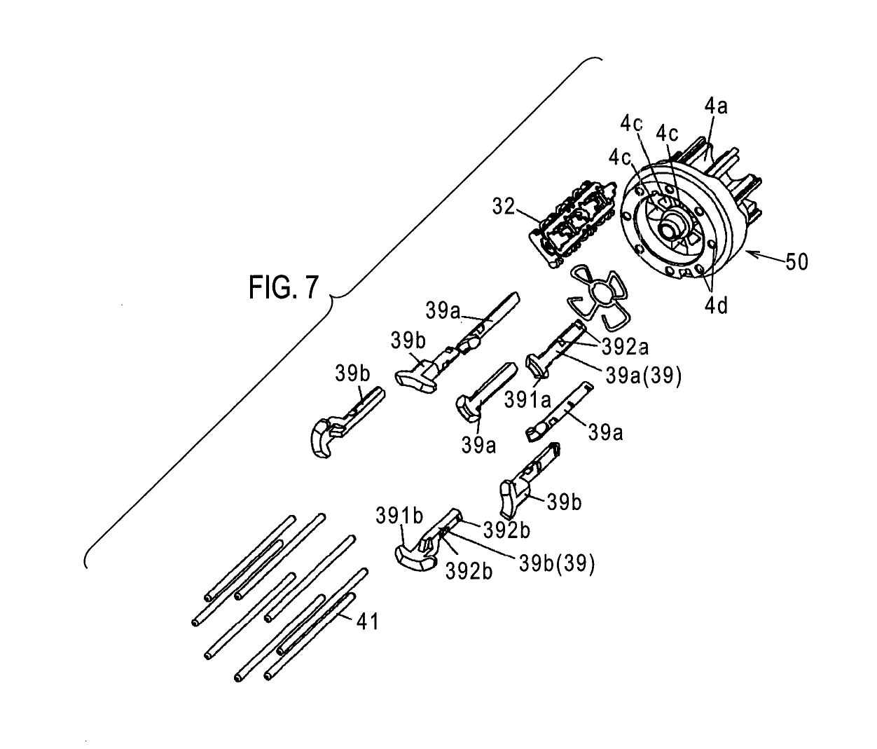

As shown in FIG. 8, the fulcrum plate 33 is formed with four rectangular holes 33c penetrating the fulcrum plate 33 in its thickness direction. A movable pawl 5 is inserted through each of the rectangular holes 33c. A base end 5c of each of the movable pawls 5 shown in FIG. 6 is fitted into grooves 392a and 392b formed in the open/close levers 39a and 39b (39), respectively, as shown in FIG. 7. With this structure, the movable pawl 5 rocks the tip end 5a around the rectangular hole 33c toward the shaft 56j in accordance with a sliding motion of the open/close levers 39a and 39b.

The fulcrum plate 33 is formed with three rectangular holes 33a between a plurality of the rectangular holes 33c. A fulcrum stop member 34 is fitted into and fixed to each of the three rectangular holes 33a. A fixing pawl 36 is sandwiched between the fulcrum stop member 34 fitted into the rectangular holes 33a on both ends and the fulcrum plate 33. Each the fixing pawl 36 is formed by bending both of substantially rectangular frame-like ends 36a toward the outer periphery of the rotation cylinder 4. As shown in FIG. 6, the both ends 36a are located adjacent the tip end 5a of the movable pawl 5. The both ends 36a of the fixing pawl 36 and the tip end 5a of the movable pawl 5 are exposed from the outer peripheral surface 10 of the rotation cylinder 4. The tip end 5a of the movable pawl 5 is switched between a state where the tip end 5a abuts against the end 36a of the fixing pawl 36 and a state where the tip end 5a is separated from the end 36a, by rocking the tip end 5a in a direction along the rotation axis Ax of the rotation cylinder 4. That is, according to the hair removing device of the present embodiment, the tip end 5a and the end 36a are opened and closed, while the rotation cylinder 4 is rotated around the rotation axis Ax. When the tip end 5a and the end 36a abut against each other, they sandwich hair therebetween, and the rotation cylinder 4 is rotated with the hair being sandwiched to pull out the hair. The outer peripheral surface 10 of the rotation cylinder 4 is formed of the outer peripheral surfaces of the end-face forming member 50 and the fulcrum stop member 34. The movable pawl 5 and the fulcrum stop member 34 have the through holes, and the fixing shafts 41 are inserted into the through holes, thereby preventing these members from falling out.

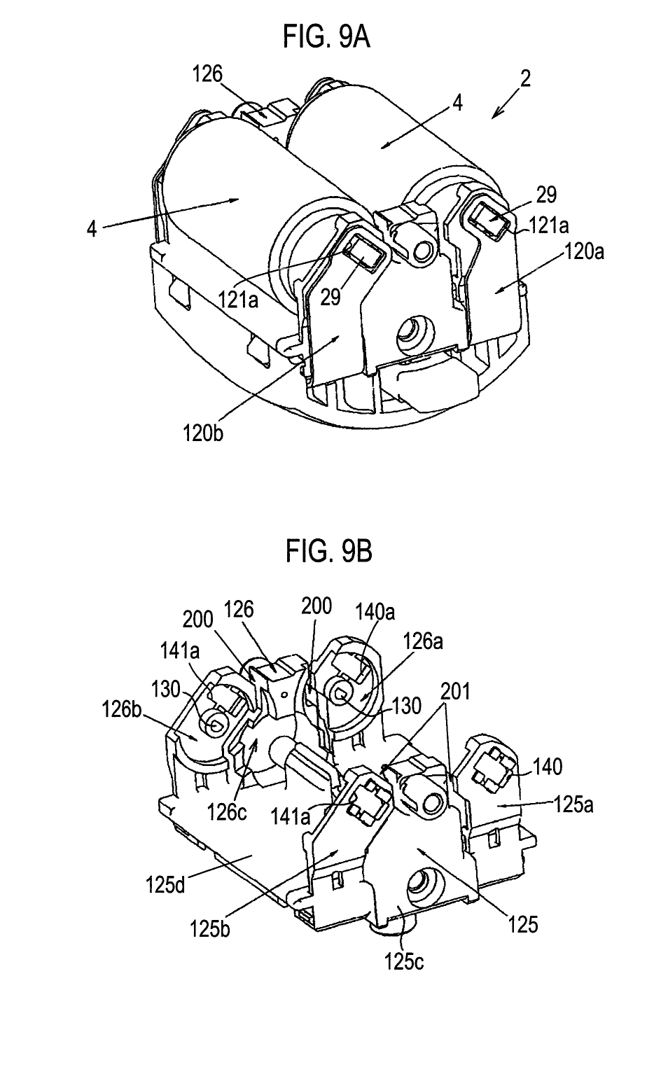

As shown in FIG. 6, the shaft 56j of the rotation cylinder 4 projects outward from the end-face forming member 50 of the rotation cylinder 4. With reference to FIG. 9B also, this projecting portions of the shafts 56j are inserted into substantially cylindrical bearings 130 respectively provided on inner sides of the pedestals 125 and 126 so that the rotation cylinder 4 is rotatably supported in a state where the rotation cylinder 4 is sandwiched on both sides by the pedestals 125 and 126. The axial length of the bearing 130 is set longer than the inserted portion of the shaft 56j, so that play is set in the rotation cylinder 4 in the axial direction. As shown in FIG. 9A, the two rotation cylinders 4 having the same structure are supported in parallel by the pedestals 125 and 126, which forms the hair removing block 2.

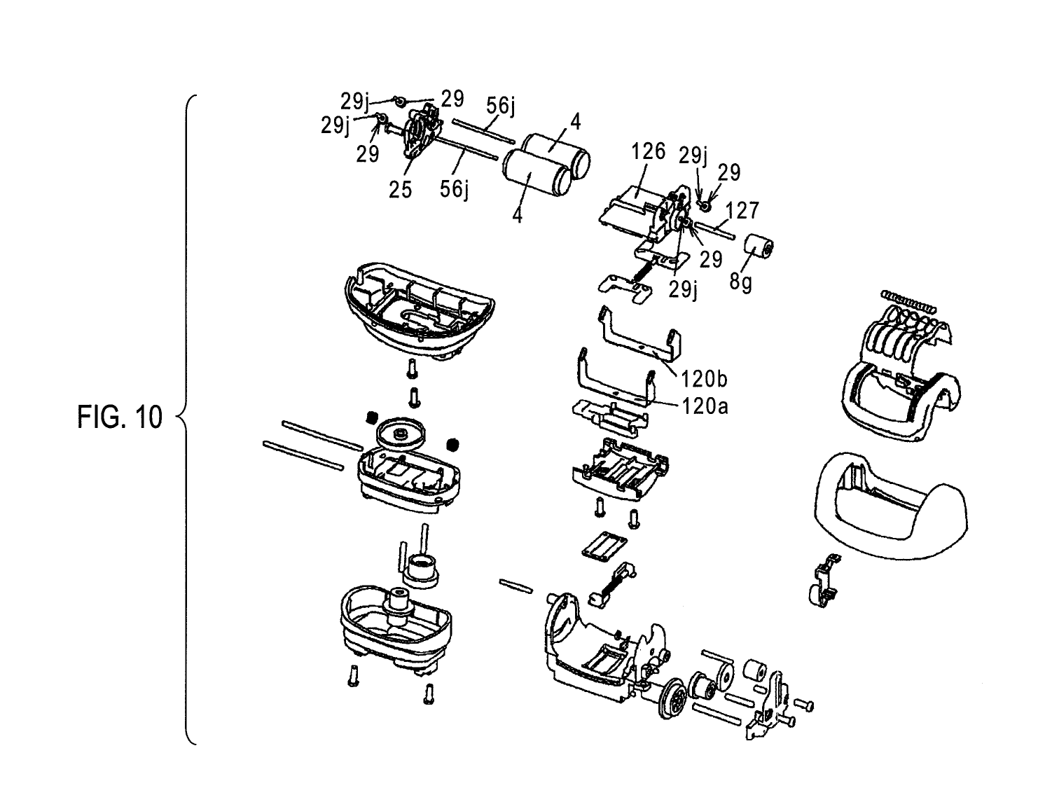

As shown in FIG. 9B, notches 201 are formed between a body 125c of the pedestal 125 and the cylinder supporting units 125a and 125b that support the rotation cylinder 4 in the pedestal 125. Notches 200 are formed between a body 126c of the pedestal 126 and cylinder supporting units 126a and 126b that support the rotation cylinder 4 in the pedestal 126. Appropriate elasticity is applied to the cylinder supporting units 125a, 125b, 126a, and 126b by these notches 201 and 200. Through holes 140a and 141a into which roller-like cams 29 are inserted are formed in the cylinder supporting units 125a, 125b, 126a, and 126b at locations opposed to the ends of the open/close levers 39 of the rotation cylinder 4. In the present embodiment, each cam 29 is provided in such an attitude that the rotation shaft 29j is located along a plane perpendicular to the axial direction of the rotation cylinder 4. Each of the through holes 140a and 141a has a shape approximately similar to a projection geometry which can be obtained by projecting the cam 29 shown in FIG. 10 in the axial direction of the rotation cylinder 4 (that is, a radial direction of the cam 29). This shape is slightly greater than the projection geometry, and has a cross shape in which rectangles having different shapes (that is, a relatively elongated rectangle corresponding to the rotation shaft 29j of the cam 29 and a relatively thick rectangle corresponding to a roller portion of the cam 29) intersect with each other. A long side of one of these rectangles being approximately similar to a cross section of the rotation shaft 29j of the cam 29 extends in a direction perpendicular to the shaft 56j of the rotation cylinder 4.

As shown in FIGS. 9A and 9B, a substantially U-shaped leaf spring 120a is fixed so as to sandwich the cylinder supporting units 125a and 126a in the present embodiment. More specifically, the leaf spring is fixed by screwing a central portion of the leaf spring 120a to a connection 125d of the body 125c of the pedestal 125 between the cylinder supporting units 125a and 126a and the cylinder supporting units 125b and 126b. Both tip ends of the leaf spring 120a can elastically be bent in a longitudinal direction (direction extending along the rotation axis Ax) of the rotation cylinder 4.

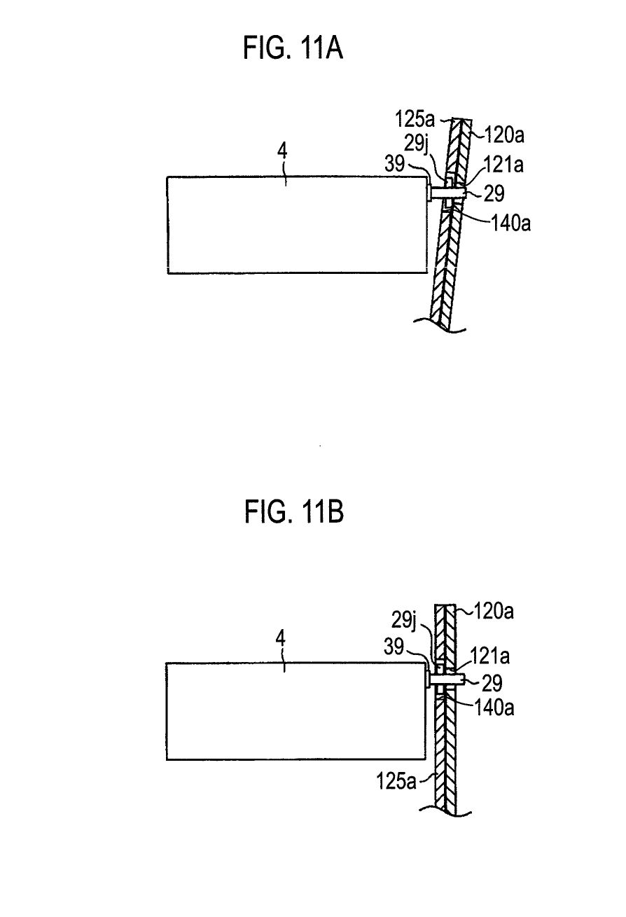

As shown in FIG. 9A, the tip ends of the leaf springs 120a and 120b are formed with substantially rectangular through holes 121a. Each of the through holes 121a has a shape approximately similar to a projection geometry which can be obtained by projecting the roller portion of the cam 29 in the axial direction of the rotation cylinder 4, and this shape is rectangular slightly greater than the projection geometry. The cams 29 are fitted into the through holes 140a and 141a of the cylinder supporting units 125a, 125b, 126a, and 126b. Unlike the through holes 140a and 141a of the cylinder supporting units 125a, 125b, 126a, and 126b, the through holes 121a of the leaf springs 120a and 120b do not have openings corresponding to the rotation shaft 29j of the cam 29, and only have openings corresponding to the roller portion of the cam 29. In this structure, as shown in FIG. 11 also, the leaf springs 120a and 120b press the rotation shafts 29j of the cams 29 toward the rotation cylinder 4, and thus the roller portions of the cams 29 are pushed against the open/close levers 39 of the rotation cylinders 4. The cams 29 are sandwiched between the leaf springs 120a and 120b and the open/close levers 39 of the rotation cylinders 4, and the roller portions of the cams 29 are inserted into the through holes 121a and supported in the axial direction of the rotation shaft 29j. As shown in FIG. 11, the cylinder supporting units 125a, 125b, 126a, and 126b are superposed on the leaf springs 120a and 120b and bent so as to follow the leaf springs 120a and 120b. Thus, the rotation shafts 29j of the cams 29 are accommodated in groove-like recesses formed so as to close thin openings corresponding to the rotation shafts 29j formed in the through holes 140a and 141a, with the leaf springs 120a and 120b.

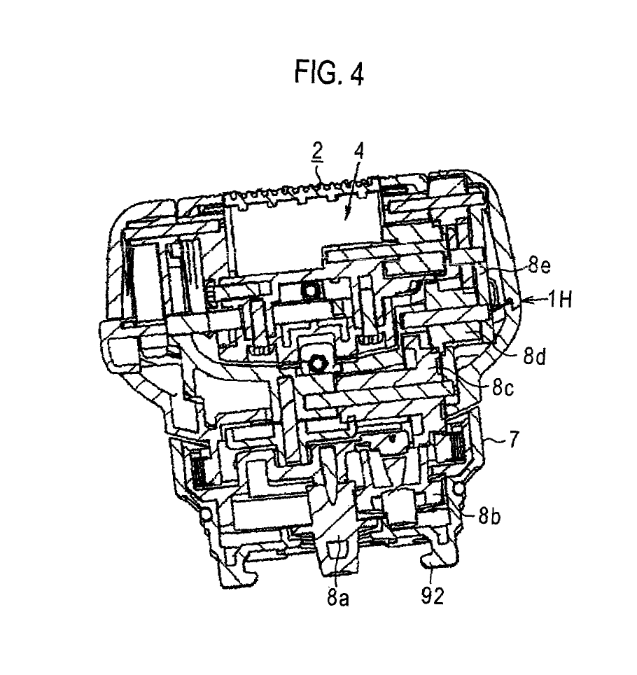

As shown in FIGS. 4 and 5B, an end of the hair removing block 2 in its longitudinal direction is formed with a gear train including gears 8a to 8f. The gears 8a to 8f are meshed with each other in this order. The gear 8a is connected to a motor (not shown) incorporated in the body, and a rotation force of the motor is transmitted to the gear 8f through the other gears. Because the gear 8f meshes with two gears 41g provided on the two rotation cylinders 4, respectively, the rotation force of the motor is transmitted to both the two rotation cylinders 4.

The hair removing head 1H constituted in this manner is mounted on the body (not shown) by a hook 92 formed on a lower portion of a body-side mounting portion 7, as shown in FIG. 4. The body is formed with a mounting hole having a shape corresponding to the hook 92, so that the hook 92 can be fitted into the mounting hole.

When the hair removing device is turned ON, the gear train including the gears 8a to 8f is rotated by the motor (not shown) incorporated in the body, and the rotation is transmitted to the two rotation cylinders 4 through the gears 41g shown in FIG. 5B. When the two rotation cylinders 4 rotate, as shown in FIG. 6, the cam 29 having a surface 29r that is pressed against a terminal 391b of the open/close lever 39 of the rotation cylinder 4 rotates, and the terminal 391b is reciprocated in the direction of the rotation axis Ax of the rotation cylinder 4. As the terminal 391b reciprocates, the open/close levers 39a and 39b rock, and the movable pawl 5 rocks the tip end 5a around the rectangular hole 33c as a fulcrum. In this manner, a state where the end 36a of the fixing pawl 36 and the tip end 5a of the movable pawl 5 abut against each other and a state where they are separated from each other are switched, that is, the end 36a of the fixing pawl 36 and the tip end 5a of the movable pawl 5 are opened and closed. When the hair removing block 2 including the two rotation cylinders 4 is brought close to a skin surface, hair growing on the skin surface enters into a gap between the end 36a of the fixing pawl 36 and the tip end 5a of the movable pawl 5 while they are separated from each other, and the hair is sandwiched. When the rotation cylinder 4 rotate with the hair being sandwiched, the hair is pulled out, that is, hair is removed.

When a long-diameter side of the cam 29 presses the open/close lever 39 as shown in FIG. 11A, because the position of the cam 29 moves toward the leaf spring 120a, the leaf spring 120a is pushed by the rotation shaft 29j of the cam 29. At that time, because the cylinder supporting unit 125a has elasticity, the cylinder supporting unit 125a follows the leaf spring 120a and moves by its own elasticity. Therefore, the leaf spring 120a and the cylinder supporting unit 125a come into contact with each other or they approach each other. When the short-diameter side of the cam 29 approaches the open/close lever 39 as shown in FIG. 11B, because the pressing force of the leaf spring 120a is weakened by the elasticity of the cylinder supporting unit 125a, a force of the cam 29 pushing the rotation axis is weakened and the open/close lever 39 is released. At that time also, the leaf spring 120a is in contact with the cylinder supporting unit 125a due to its own elasticity. Therefore, according to the present embodiment, the leaf spring 120a and the cylinder supporting unit 125a can always be in contact with or approach each other. This structure can be applied not only to the leaf spring 120a and the cylinder supporting unit 125a, but also to the leaf spring 120a and the cylinder supporting unit 126a, the leaf spring 120b and the cylinder supporting unit 125b, and the leaf spring 120b and the cylinder supporting unit 126b.

In the present embodiment, because the leaf springs 120a and 120b and the cylinder supporting units 125a, 125b, 126a, and 126b corresponding thereto can always be in contact with or approach each other, it is possible to suppress a noise caused by collision between the leaf springs 120a and 120b and the corresponding cylinder supporting units 125a, 125b, 126a, and 126b.

In the present embodiment, the cylinder supporting units 125a, 125b, 126a, and 126b can elastically be bent, so that the cylinder supporting units 125a, 125b, 126a, and 126b follow the corresponding leaf springs 120a and 120b. Thus, the cylinder supporting units 125a and 125b as well as 126a and 126b can relatively easily follow the leaf springs 120a and 120b without using a special interlocking device.

In the present embodiment, the notches 200 and 201 are formed between the cylinder supporting units 125a, 125b, 126a, and 126b and the bodies 125c and 126c of the pedestals 125 and 126. With this, appropriate elasticity can more easily and inexpensively be given to each of the cylinder supporting units 125a, 125b, 126a, and 126b.

In the present embodiment, the cam 29 of the hair removing device has the roller-like shape. If the roller-shaped cam 29 is used, the cam 29 can be formed of more inexpensive member having higher general versatility.

In the present embodiment, the cams 29 are inserted into the through holes 140a and 141a formed in the cylinder supporting units 125a, 125b, 126a, and 126b, the rotation shafts 29j are pressed by the springs 120a and 120b, and the surface 29r of the cam 29 is pressed against the terminal 391b of the open/close lever 39, so that the cam 29 is rotatably supported. Thus, it is unnecessary to form a special bearing, and the cam can be supported and pressed with a relatively simple structure.

The present embodiment can be modified as follows.

Although the leaf spring is used as a spring biasing the cam in the above embodiment, this spring is not limited to the leaf spring, and a coil spring can be used for biasing the cam for example.

Although the notch is provided between the cylinder supporting unit and the body of the pedestal to apply the elasticity to the cylinder supporting unit in the above embodiment, other methods can be used instead. A part or the entire pedestal including the cylinder supporting unit can be formed of elastic member such as rubber.

Although the roller-shaped cam is used in the above embodiment, the cam can have other shapes. For example, a flat face groove cam or conjugated cam can be used.

A rocking function in a direction perpendicular to the rotation cylinder shaft can be added to the hair removing block of the hair removing device according to the above embodiment. If the hair removing block has such a function, and when the hair removing block is pressed against a skin, the hair removing block can rock in accordance with an uneven shape of the skin surface. Therefore, the pressing force of the hair removing block against a skin is substantially equalized and thus, damage on a skin can be reduced and the hair removing efficiency is also enhanced. As the rocking function, for example, a technique described in Japanese Patent Application Laid-open No. 2006-175026 can be applied.

In the hair removing device according to the modification, a comb that rocks in association with the hair removing block can be provided between the two rotation cylinders. When the hair removing block is pressed against a skin surface, the comb first comes into contact with the skin so that a root portion of a hair is pressed to squeeze the hair, and the hair can efficiently be removed. A combing effect can be added, and touch on a skin can be moderated.

The hair removing device according to the above embodiment can be provided with a function of detaching the hair removing head. If such a function is added, the cleaning performance of the hair removing head is enhanced, and the hair removing head can be exchanged. It is possible to change the kinds of hair removing heads in accordance with hair, or only a worn hair removing head can be replaced. As the detaching function, for example, a technique described in Japanese Patent Application Laid-open No. 2003-275021 can be applied.

The present invention can be carried out as a hair removing device that pulls out hair from a skin surface.

### CROSS REFERENCE TO RELATED APPLICATION

This application is based upon and claims the benefit of priority from a Japanese Patent Application No. 2007-169277, filed on Jun. 27, 2007; the entire contents of which are incorporated herein by reference.

What is claimed is:

1. A hair removing device comprising:
a substantially cylindrical rotation cylinder that rotates around a rotation axis;
a fixing pawl that is fixed to the rotation cylinder and that has a tip end exposed from an outer periphery of the rotation cylinder;
a movable pawl that can rock in an axial direction of the rotation axis, that has a tip end exposed from the outer periphery of the rotation cylinder, and that is configured such that a state where the tip end of the movable pawl abuts against the tip end of the fixing pawl and a state where these tip ends are separated are switched by a rocking motion of the movable pawl;
an open/close lever that is inserted into the rotation cylinder such that the open/close lever can reciprocate in an axial direction of the rotation cylinder, and that rocks the movable pawl;
a cam that reciprocates the open/close lever in the axial direction while rotating around the rotation axis,
a pedestal that rotatably supports the rotation cylinder and the cam; and
a spring that biases the cam toward the open/close lever to press the cam against the open/close lever, wherein
a cylinder supporting unit as a portion of the pedestal that supports the rotation cylinder and the cam can rock in association with the spring with respect to a body of the pedestal.

2. The hair removing device according to claim 1, wherein the cylinder supporting unit has elasticity.

3. The hair removing device according to claim 2, wherein a notch is formed between the cylinder supporting unit and the body.

4. The hair removing device according to claim 1, wherein the cam has a roller-like shape.

5. The hair removing device according to claim 4, wherein
the cylinder supporting unit is formed with a through hole in which a rotation shaft of the cam is rotatably accommodated, and
the rotation shaft is biased by the spring from a side opposite from the open/close lever.
